(19) United States
(12) Patent Application Publication
(10) Pub. No.: US 2025/0000424 A1
(43) Pub. Date: Jan. 2, 2025

(54) Method and System for Measuring and Displaying Biosignal Data to a Wearer of a Wearable Article

(71) Applicant: Prevayl Innovations Limited, Manchester, Greater Manchester (GB)

(72) Inventor: Connor David Driscoll, Manchester, Greater Manchester (GB)

(21) Appl. No.: 18/706,070

(22) PCT Filed: Nov. 14, 2022
(86) PCT No.: PCT/GB2022/052884

§ 371 Date: Apr. 30, 2024

(30) Foreign Application Priority Data
Nov. 17, 2021 (GB) 2116581.6

(51) Int. Cl. A61B 5/364; A61B 5/00; A61B 5/024

(52) U.S. Cl. CPC A61B 5/364; A61B 5/02438; A61B 5/7214

(57) ABSTRACT

An electronics module (100) receives biosignals such as ECG signals from sensors on a wearable article (200) and processes these signals to provide data and information to a user electronic device (300). The electronics module is operable to detect when the processed ECG output includes spurious or otherwise anomalous peaks. Where an anomalous peak is detected, the ECG output is corrected by sequentially applying a series of correcting steps and analysing each one to determine which correcting step provides the least anomalous one. Anomalous peaks are detected by looking at the heart rate variability and determining that an anomaly is present when the rate of change of heart rate is above a threshold level. The threshold level is configured depending upon certain criteria such as the activity level of a person whose heart rate is being monitored or selected subject-specific criteria. Thus, an improved ECG output is produced, which, when displayed to a wearer of the electronics module is more informative and understandable.

The present invention is directed towards a system comprising an electronics module, a wearable article in the form of a garment, and a user electronic device communicatively coupled to the electronics module. More particularly, the wearable article comprises a biosignal measuring apparatus for sensing biosignals from a wearer of the wearable article, and which incorporates a sensor assembly and the electronics module. The electronics module is arranged to transmit biosignal data to the user electronics module or other remote device. The present invention is also directed towards a controller for an electronics module and a wearable article incorporating an electronics module.

### BACKGROUND

Wearable articles, such as garments, incorporating sensors are wearable electronics used to measure and collect information from a wearer. It is advantageous to measure biosignals of the wearer during exercise, or other scenarios.

It is known to provide a garment, or other wearable article, to which an electronic device (i.e. an electronic module, and/or related components) is attached in a prominent position, such as on the chest or between the shoulder blades. Advantageously, the electronic device is a detachable device. The electronic device is configured to process the incoming signals, and the output from the processing is stored and/or displayed to a user in a suitable way

A sensor senses a biosignal such as electrocardiogram (ECG) signals and the biosignals are coupled to the electronic device, via an interface.

The sensors may be coupled to the interface by means of conductors which are connected to terminals provided on the interface to enable coupling of the signals from the sensor to the interface.

Electronics modules for wearable articles such as garments are known to communicate with user electronic devices over wireless communication protocols such as Bluetooth® and Bluetooth® Low Energy. These electronics modules are typically removably attached to the wearable article, interface with internal electronics of the wearable article, and comprise a Bluetooth® antenna for communicating with the user electronic device.

The electronic device includes drive and sensing electronics comprising components and associated circuitry, to provide the required functionality.

The drive and sensing electronics include a power source to power the electronic device and the associated components of the drive and sensing circuitry.

ECG sensing is used to provide a plethora of information about a person's heart. It is one of the simplest and oldest techniques used to perform cardiac investigations. In its most basic form, it provides an insight into the electrical activity generated within heart muscles that changes over time. By detecting and amplifying these differential biopotential signals, a lot of information can be gathered quickly, including the heart rate. Among professional medical staff, individual signals have names such as “the QRS complex,” which is the largest part of an ECG signal and is a collection of Q, R, and S signals, including the P and T waves.

Whilst lay persons may not be aware of the clinical aspects and significance of an ECG signal trace, lay persons would usually recognise the general form of such a signal trace, if only as a measure of heart rate.

Typically, the detected ECG signals can be displayed as a trace to a user for information. The user may be a clinician who is looking to assess cardiac health or may be a lay user using the electronics module has a fitness or health and wellness assessment device. A typical ECG waveform or trace 800 is illustrated in FIG. 1 showing the QRS complex.

FIG. 2 shows an ECG waveform of two successive heartbeats. The time between successive heart beats is known as the inter-beat interval (IBI). This usually measured as the time between successive R peaks of the ECG waveform i.e. the R-R interval, although it could be measured as the time between other peaks of the QRS complex such as the differences between S troughs (the S-S interval) or P peaks (the P-P interval). In the present embodiment of the invention, the R-R interval is measured and used to calculate the IBI. This time difference is usually expressed in milliseconds. IBI values represent the time between successive heartbeats.

The trace can be displayed on a user electronic device such as a mobile phone.

An individual's heart rate will change depending upon, for example, whether a person is at rest or is active.

Occasionally, when monitoring a person's hear rate, an R peak may go undetected, or an additional or spurious beat may be detected. This may be due to a fault in the sensing of the biosignal, noise artefacts or sometimes is due to an individual's heart producing an extra beat or missing a bit. This is referred to as an ectopic heartbeat.

The ability to detect and correct for anomalous heart traces is therefore important in providing reliable heart rate data to a user.

Methods for detecting and correcting for anomalous heart rates are known. One method is described, for example, in “Analysis of heart rate variability in the presence of ectopic beats using the heart timing signal” by Mateo, J. and Laguna P. in the IEEE Transactions on Biomedical Engineering, Volume 50, No. 3, March 2013.

Such analysis is predicated on the understanding that heart rate is incapable of rapid changes from its current value, that is to say, that the rate of change of heart rate will normally be below some threshold.

An object of the present invention is to provide an improved method and system for measuring and displaying biosignal data to a wearer of a wearable article and also a user electronic device for a wearable article so as to provide for a better user experience to the extent that spurious, missing and otherwise anomalous heart beats are detected and corrected for in a timely manner such that valid insights can be provided to a user.

### SUMMARY

According to an aspect of the present invention, there is provided a computer-implemented method of correcting an electrocardiogram output for anomalous heart beats, the method comprising:
obtaining a series heartrate values for the electrocardiogram output;
determining when the rate of change of the heartrate values exceeds a predetermined threshold value; and, in response to the predetermined threshold value being exceeded, sequentially applying one or more correcting steps to a region of the electrocardiogram output around a selected heartbeat where the rate of change of heartrate values occurred;
obtaining information indicative of the likely signal-to-noise ratio of the electrocardiogram output; and
configuring the predetermined threshold value based on the likely signal-to-noise ratio

The predetermined threshold value may be configured to be a first value when the likely signal-to-noise ratio is above a first predetermined value.

The predetermined threshold value may configured to be a second value when the likely signal-to-noise ratio is above a second predetermined value.

The obtained information may relate to the activity level of a person whose electrocardiogram output is being monitored.

Alternatively, the obtained information may relate to a characteristic of a person whose electrocardiogram output is being monitored. The characteristics may be gender, weight, and body fat level of a person who electrocardiogram is being monitored.

The method may further comprise:
determining the rate of change of heartrate values at the selected region for each of the applied correcting steps; and
choosing a correcting step corresponding to the lowest of the rate of change of heartrate values at the selected region as a permanent correction for the electrocardiogram output.

The method may further comprise: obtaining a series heartrate values for the electrocardiogram output; determining when the rate of change of the heartrate values exceeds a predetermined threshold value; and, in response to the predetermined threshold value being exceeded, sequentially applying one or more correcting steps to a region of the electrocardiogram output around a selected heartbeat where the rate of change of heartrate values occurred; determining the rate of change of heartrate values at the selected region for each of the applied correcting steps; and choosing a correcting step corresponding to the lowest of the rate of change of heartrate values at the selected region as a permanent correction for the electrocardiogram output.

The step of determining the rate of change of heartrate values for each of the applied correcting steps may comprise. determining the rate of change of the heartrate values for three consecutive heartbeats in the region of the electrocardiogram output around the selected heartbeat.

The method may further include summing the rate of change of the heartrate values determined for each of the three consecutive heartbeats.

The lowest of the rate of change of heartrate values at the selected region may be the lowest of the summed rate of change of heartrate values.

After applying a one of the one or more correcting steps in a region of the electrocardiogram output around a selected heartbeat, and determining the rate of change of heartrate values, the applied one of the one or more correcting steps may be removed before applying another one of the one or more correcting steps.

A chosen correction step may be only applied as a permanent correction for the electrocardiogram output if the rate of change heart rate value is below the predetermined threshold value.

The region of the electrocardiogram output around a selected heartbeat may comprise the selected heartbeat, and heartbeats immediately before and after the selected heartbeat, and the correcting step is one or more of:
a) Removing the selected heartbeat;
b) Removing heartbeat immediately after the selected heartbeat;
c) Inserting a heartbeat between the selected heartbeat and the heartbeat immediately before the selected heartbeat;
d) Inserting a heartbeat between the selected heartbeat and the heartbeat immediately after the selected heartbeat;
e) Moving the selected heartbeat to an intermediate position between the heartbeats immediately before and immediately after the selected heartbeat; and
f) Move the heartbeat after the selected heartbeat to an intermediate position between the selected heart beat and the second heartbeat immediately after the selected heartbeat.

Alternatively, the correcting step may be all of:
a) Removing the selected heartbeat;
b) Removing heartbeat immediately after the selected heartbeat;
c) Inserting a heartbeat between the selected heartbeat and the heartbeat immediately before the selected heartbeat;
d) Inserting a heartbeat between the selected heartbeat and the heartbeat immediately after the selected heartbeat;
e) Moving the selected heartbeat to an intermediate position between the heartbeats immediately before and immediately after the selected heartbeat; and
f) Move the heartbeat after the selected heartbeat to an intermediate position between the selected heart beat and the second heartbeat immediately after the selected.

The rate of change heart rate value is derived from the equation:

$$\vert {r}_{k}^{′} \vert = 2 \vert \frac{{t}_{k - 1} - 2 {t}_{k} + {t}_{k + 1}}{( {t}_{k - 1} - {t}_{k} ) ( {t}_{k - 1} - {t}_{k + 1} ) ( {t}_{k} - {t}_{k + 1} )} \vert$$

where rₖ′ is the rate of change of the heart rate, at the k^{th} heartbeat,
tₖ is the time of the k^{th} heartbeat, and
tₖ₋₁ and tₖ₊₁ are the times of the (k−1)^{th} and (k+1)^{th} heartbeat respectively either side of the k^{th} heartbeat.

The predetermined threshold value may be in the range 1×10⁻⁵ to 1×10⁻⁷ and more preferably of the order of 10⁻⁶.

The information obtains indicative of the likely signal-to-noise ratio of the ECG signal may be subject specific information and this can be used to configure the threshold value.

The value of U may be selected to be a value that is known to be significantly higher than any possible rate of change of heart rate where rₖ′.

The step of obtaining a series heartrate values for the electrocardiogram may comprise a step of sampling a series of heart beats in the electrocardiogram output and determining the heart rate values from the sampled heart beats.

The step of obtaining a series heartrate values for the electrocardiogram may comprise sampling a first set of heart beats and a second set of heart beats, removing the last two heart beats from the first set of heart beats, removing the last two heart beats of the second set of heart beats and combining the last two heart beats from the first set of heart beats with the remaining heart beats from the second set of heart beats.

The electrocardiogram output may be sampled in windows of a fixed size. This may be 2048 samples. The window may be a four second window.

In another aspect of the present invention, there is provided a computer-readable medium having instructions recorded thereon which, when executed by a processor, cause the processor to perform the method as recited in the first aspect of the invention.

In a third aspect of the present invention, there is provided a system for correcting an electrocardiogram output for anomalous heart beats, the system comprising a controller and a memory, the memory storing instructions which when executed by the controller cause the controller to perform operations comprising:
obtaining a series heartrate values for the electrocardiogram output;
determining when the rate of change of the heartrate values exceeds a predetermined threshold value; and, in response to the predetermined threshold value being exceeded,
sequentially applying one or more correcting steps to a region of the electrocardiogram output around a selected heartbeat where the rate of change of heartrate values occurred;
obtaining information indicative of the likely signal-to-noise ratio of the electrocardiogram output; and
configuring the predetermined threshold value based on the likely signal-to-noise ratio,
The predetermined threshold value may be configured to be a first value when the likely signal-to-noise ratio is above a first predetermined value.

The predetermined threshold value may be configured to be a second value when the likely signal-to-noise ratio is above a second predetermined value.

The obtained information may relate to the activity level of a person whose electrocardiogram output is being monitored.

The obtained information may relate to a characteristic of a person whose electrocardiogram output is being monitored.

The memory may store further instructions which when executed by the controller cause the controller to perform operations comprising:
determining the rate of change of heartrate values at the selected region for each of the applied correcting steps; and
choosing a correcting step corresponding to the lowest of the rate of change of heartrate values at the selected region as a permanent correction for the electrocardiogram output.

In a fourth aspect of the present invention, there is provided an electronics module comprising a controller and a memory coupled to the controller, the controller being arranged to receive signals from a sensor arrangement on a wearable article, wherein the controller is configured to obtain electrocardiogram output for a wearer of the wearable article from the sensor arrangement, the controller being further configured to:
obtain a series heartrate values for the electrocardiogram output;
determine when the rate of change of the heartrate values exceeds a predetermined threshold value; and, in response to the predetermined threshold value being exceeded,
sequentially apply one or more correcting steps to a region of the electrocardiogram output around a selected heartbeat where the rate of change of heartrate values occurred;
obtain information indicative of the likely signal-to-noise ratio of the electrocardiogram output; and
configure the predetermined threshold value based on the likely signal-to-noise ratio,

The predetermined threshold value may be configured to be a first value when the likely signal-to-noise ratio is above a first predetermined value.

The predetermined threshold value may be configured to be a second value when the likely signal-to-noise ratio is above a second predetermined value.

The obtained information may relate to the activity level of a person whose electrocardiogram output is being monitored.

The obtained information may relate to a characteristic of a person whose electrocardiogram output is being monitored.

The controller may be further configured to:
determine the rate of change of heartrate values at the selected region for each of the applied correcting steps; and
choose a correcting step corresponding to the lowest of the rate of change of heartrate values at the selected region as a permanent correction for the electrocardiogram output.

The controller may be further configured to sum the rate of change of the heartrate values determined for each of the three consecutive heartbeats.

The controller may be further configured to select the lowest of the summed rate of change of heartrate values as the lowest of the rate of change of heartrate values at the selected region.

The controller may be further configured, after applying a one of the one or more correcting steps in a region of the electrocardiogram output around a selected heartbeat and determining the rate of change of heartrate values, to remove the applied one of the one or more correcting steps before applying another one of the one or more correcting steps.

The controller may be further configured to only apply a chosen correction step as a permanent correction for the electrocardiogram output if the rate of change heart rate value is below the predetermined threshold value.

The controller may be configured to sample a first set of heart beats and a second set of heart beats, to remove the last two heart beats from the first set of heart beats, to remove the last two heart beats of the second set of heart beats and to combine the last two heart beats from the first set of heart beats with the remaining heart beats from the second set to derive the sampled heart beats.

The correcting step applied by the controller may be one or more of:
g) Removing the selected heartbeat;
h) Removing heartbeat immediately after the selected heartbeat;
i) Inserting a heartbeat between the selected heartbeat and the heartbeat immediately before the selected heartbeat;
j) Inserting a heartbeat between the selected heartbeat and the heartbeat immediately after the selected heartbeat;
k) Moving the selected heartbeat to an intermediate position between the heartbeats immediately before and immediately after the selected heartbeat; and
l) Move the heartbeat after the selected heartbeat to an intermediate position between the selected heart beat and the second heartbeat immediately after the selected heartbeat.

Alternatively, the correcting step applied by the controller may be all of:
a) Removing the selected heartbeat;
b) Removing heartbeat immediately after the selected heartbeat;
c) Inserting a heartbeat between the selected heartbeat and the heartbeat immediately before the selected heartbeat;
d) Inserting a heartbeat between the selected heartbeat and the heartbeat immediately after the selected heartbeat;
e) Moving the selected heartbeat to an intermediate position between the heartbeats immediately before and immediately after the selected heartbeat; and
f) Move the heartbeat after the selected heartbeat to an intermediate position between the selected heart beat and the second heartbeat immediately after the selected heartbeat.

The controller may be configured to derive the rate of change heart from the equation:

$$\vert {r}_{k}^{′} \vert = 2 \vert \frac{{t}_{k - 1} - 2 {t}_{k} + {t}_{k + 1}}{( {t}_{k - 1} - {t}_{k} ) ( {t}_{k - 1} - {t}_{k + 1} ) ( {t}_{k} - {t}_{k + 1} )} \vert$$

where rₖ′ is the rate of change of the heart rate, at the k^{th} heartbeat,
tₖ is the time of the k^{th} heartbeat, and
tₖ₋₁ and tₖ₊₁ are the times of the (k−1)^{th} and (k+1)^{th} heartbeat respectively either side of the k^{th} heartbeat.

The controller may be configured to configure the predetermined threshold value.

The predetermined threshold hold value may be in the range 1×10⁻⁵ to 1×10⁻⁷ and more preferably of the order of 10⁻⁶.

The predetermined threshold value may be in the range 1×10⁻⁵ to 1×10⁻⁷ and more preferably of the order of 10⁻⁶.

The information obtains indicative of the likely signal-to-noise ratio of the ECG signal may be subject specific information and this can be used to configure the threshold value.

The value of U may be selected to be a value that is known to be significantly higher than any possible rate of change of heart rate where rₖ′.

The controller may be configured to obtain a series heartrate values for the electrocardiogram by sampling a series of heart beats in the electrocardiogram output and determining the heart rate values from the sampled heart beats.

The controller may be configured to obtaining the series heartrate values for the electrocardiogram by sampling a first set of heart beats and a second set of heart beats, removing the last two heart beats from the first set of heart beats, removing the last two heart beats of the second set of heart beats and combining the last two heart beats from the first set of heart beats with the remaining heart beats from the second set of heart beats.

The electrocardiogram output may be sampled in windows of a fixed size. This may be 2048 samples. The window may be a four second window.

The electronics module may comprise an analogue to digital front end and the controller may be configured to derive the heart rate values from the analogue to digital front end.

The present invention provides an improved computer-implemented method of correcting electrocardiogram insights that are configured to be provided to a user of an electronics module which can be coupled to sensors configured to detect biosignals of the wearer. The invention enables spurious heart beats to be corrected for so that a corrected ECG data and or ECG trace can be displayed to the wearer. This provides insights which are more readily understood and informative by the wearer without causing alarm, for example, where the beats are irregular.

According to a fifth aspect of the present invention, there is provided a computer-implemented method. The method comprises obtaining biosignal data for the wearer of a wearable article. The method further comprises; obtaining activity data; determining an activity level for a wearer of the wearable article from the obtained activity data; determining if the activity level is above or below a predetermined threshold and, when the activity level is below the predetermined threshold, retrieving insight data for the obtained the obtained biosignal data from a first insight data generator when the activity level is below the predetermined threshold, retrieving insight data for the obtained biosignal data from a second insight data generator.

The insight data is preferably ECG data and more preferably the inter-beat interval for an ECG.

The method may further comprises providing an electronics module and the insight data may be obtained from an analogue to digital front end of the electronics module. Alternatively, the insight data may be determined by a controller of the electronics module.

The activity data may be obtained from the electronics module. Alternatively, activity data may be obtained from a user electronic device.

Obtaining activity may comprise obtaining motion data for the wearer of the wearable article. Alternatively, obtaining activity data comprises obtaining orientation data for the wearer of the wearable article.

The method may be performed by a controller for a user electronic device, the user electronic device further including an interface, coupled to the controller, and arranged to receive signals from an electronics module for a wearable article.

### BRIEF DESCRIPTION OF THE DRAWINGS

Examples of the present disclosure will now be described with reference to the accompanying drawings, in which:

FIG. 1 illustrates a signal trace for an ECG signal;

FIG. 2 illustrates an ECG waveform that includes electrical signals for two successive heartbeats;

FIG. 3 shows a schematic diagram for an example system according to aspects of the present disclosure;

FIG. 4 illustrates a user electronic device displaying an ECG signal trace;

FIG. 5 shows a schematic diagram for an example electronics module according to aspects of the present disclosure;

FIG. 6 shows a schematic diagram for another example electronics module according to aspects of the present disclosure;

FIG. 7 shows a schematic diagram for an example analogue-to-digital converter used in the example electronics module of FIGS. 4 and 5 according to aspects of the present disclosure;

FIG. 8 shows a detailed schematic diagram of the components of an example user electronics device according to aspects of the present disclosure;

FIG. 9 is a schematic illustration of an ECG trace showing the inter beat interval;

FIG. 10 is a schematic representation of a window sample of heart beats as used in an example of the present invention;

FIG. 11 is a schematic illustrations of a three-window sample of heart beats as used in an example of the present invention;

FIG. 12 is a schematic representation of anomaly correction steps of an example of a method of the present invention;

FIG. 13A to 13D shows a flow diagram for an example method according to aspects of the present disclosure;

FIG. 14 is a flow diagram for an example method for calculating the inter beat interval;

FIG. 15 is a flow diagram for an example method for selecting an R-R detection method; and

FIG. 16 is a flow diagram for another example method for selecting an R-R detection method

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings but are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

“Wearable article” as referred to throughout the present disclosure may refer to any form of device interface which may be worn by a user such as a smart watch, necklace, garment, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, garment brassiere, shorts, pants, arm or leg sleeve, vest, jacket/coat, glove, armband, underwear, headband, hat/cap, collar, wristband, stocking, sock, or shoe, athletic clothing, personal protective equipment, including hard hats, swimwear, wetsuit or dry suit. The term “wearer” includes a user who is wearing, or otherwise holding, the wearable article.

The type of wearable garment may dictate the type of biosignals to be detected. For example, a hat or cap may be used to detect electroencephalogram or magnetoencephalogram signals.

The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the application. Silk may also be used as the natural fibre. Cellulose, wool, hemp and jute are also natural fibres that may be used in the wearable article/garment. Polyester, polycotton, nylon and viscose are synthetic fibres that may be used in the wearable article/garment.

The garment may be a tight-fitting garment. Beneficially, a tight-fitting garment helps ensure that the sensor devices of the garment are held in contact with or in the proximity of a skin surface of the wearer. The garment may be a compression garment. The garment may be an athletic garment such as an elastomeric athletic garment.

The garment has sensing units provided on an inside surface which are held in close proximity to a skin surface of a wearer wearing the garment. This enables the sensing units to measure biosignals for the wearer wearing the garment.

The sensing units may be arranged to measure one or more biosignals of a wearer wearing the garment.

“Biosignal” as referred to throughout the present disclosure may refer to signals from living beings that can be continually measured or monitored. Biosignals may be electrical or non-electrical signals. Signal variations can be time variant or spatially variant.

Sensing components may be used for measuring one or a combination of bioelectrical, bioimpedance, biochemical, biomechanical, bioacoustics, biooptical or biothermal signals of the wearer 600. The bioelectrical measurements include electrocardiograms (ECG), electrogastrograms (EGG), electroencephalograms (EEG), and electromyography (EMG). The bioimpedance measurements include plethysmography (e.g., for respiration), body composition (e.g., hydration, fat, etc.), and electroimpedance tomography (EIT). The biomagnetic measurements include magnetoneurograms (MNG), magnetoencephalography (MEG), magnetogastrogram (MGG), magnetocardiogram (MCG). The biochemical measurements include glucose/lactose measurements which may be performed using chemical analysis of the wearer 600's sweat. The biomechanical measurements include blood pressure. The bioacoustics measurements include phonocardiograms (PCG). The biooptical measurements include orthopantomogram (OPG). The biothermal measurements include skin temperature and core body temperature measurements.

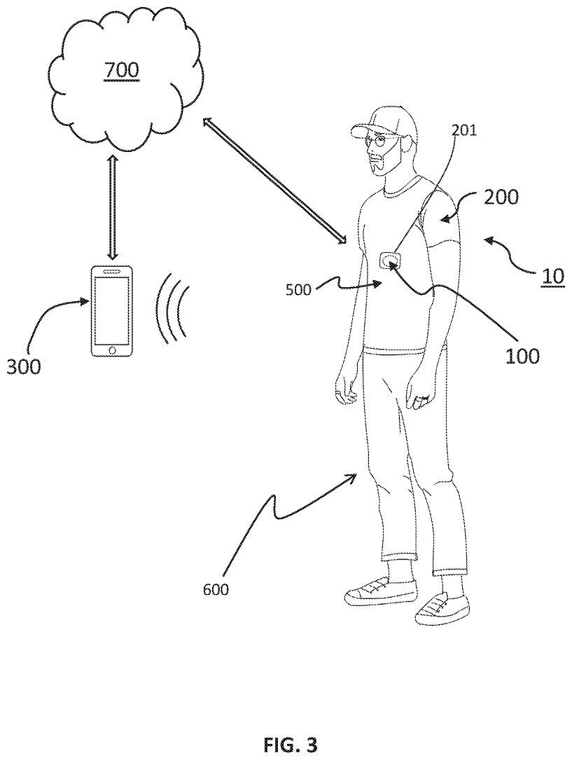

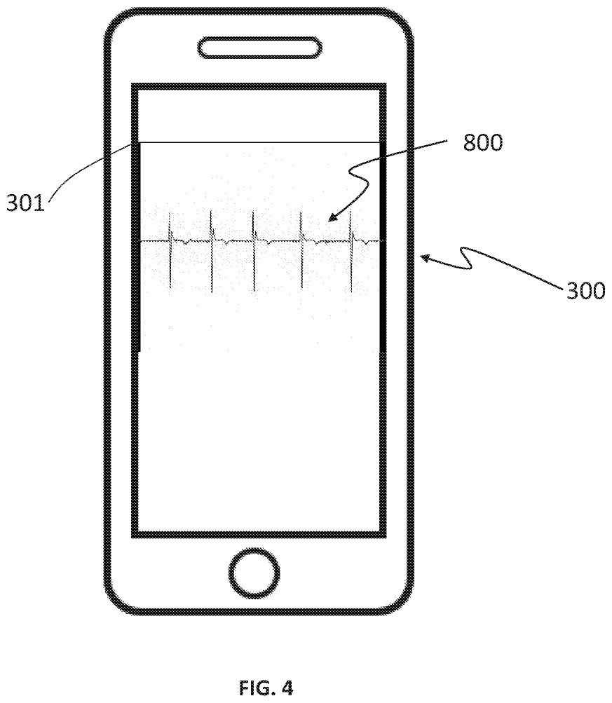

Referring to FIGS. 3 to 8, there is shown an example system 10 according to aspects of the present disclosure. The system 10 comprises an electronics module 100, a wearable article in the form of a garment 200, and a user electronic device 300. The garment 200 is worn by a user who in this embodiment is a wearer 600 of the garment 200.

The electronics module 100 is arranged to integrate with sensing units 400 incorporated into the garment 200 to obtain signals from the sensing units 400.

The electronics module 100 and the wearable article 200 and including the sensing units 400 comprise a wearable assembly 500.

The sensing units 400 comprise one or more sensors 209, 211 with associated conductors 203, 207 and other components and circuitry. The electronics module 100 is further arranged to wirelessly communicate data to the user electronic device 300. Various protocols enable wireless communication between the electronics module 100 and the user electronic device 300. Example communication protocols include Bluetooth®, Bluetooth® Low Energy, and near-field communication (NFC).

The garment 200 has an electronics module holder in the form of a pocket 201. The pocket 201 is sized to receive the electronics module 100. When disposed in the pocket 201, the electronics module 100 is arranged to receive sensor data from the sensing units 400. The electronics module 100 is therefore removable from the garment 200.

The present disclosure is not limited to electronics module holders in the form pockets.

Alternatively, the electronics module 100 may be configured to be releasably mechanically coupled to the garment 200. The mechanical coupling of the electronic module 100 to the garment 200 may be provided by a mechanical interface such as a clip, a plug and socket arrangement, etc. The mechanical coupling or mechanical interface may be configured to maintain the electronic module 100 in a particular orientation with respect to the garment 200 when the electronic module 100 is coupled to the garment 200. This may be beneficial in ensuring that the electronic module 100 is securely held in place with respect to the garment 200 and/or that any electronic coupling of the electronic module 100 and the garment 200 (or a component of the garment 200) can be optimized. The mechanical coupling may be maintained using friction or using a positively engaging mechanism, for example.

Beneficially, the removable electronic module 100 may contain all the components required for data transmission and processing such that the garment 200 only comprises the sensing units 400 e.g. the sensors 209, 211 and communication pathways 203, 207. In this way, manufacture of the garment 200 may be simplified. In addition, it may be easier to clean a garment 200 which has fewer electronic components attached thereto or incorporated therein. Furthermore, the removable electronic module 100 may be easier to maintain and/or troubleshoot than embedded electronics. The electronic module 100 may comprise flexible electronics such as a flexible printed circuit (FPC).

The electronic module 100 may be configured to be electrically coupled to the garment 200.

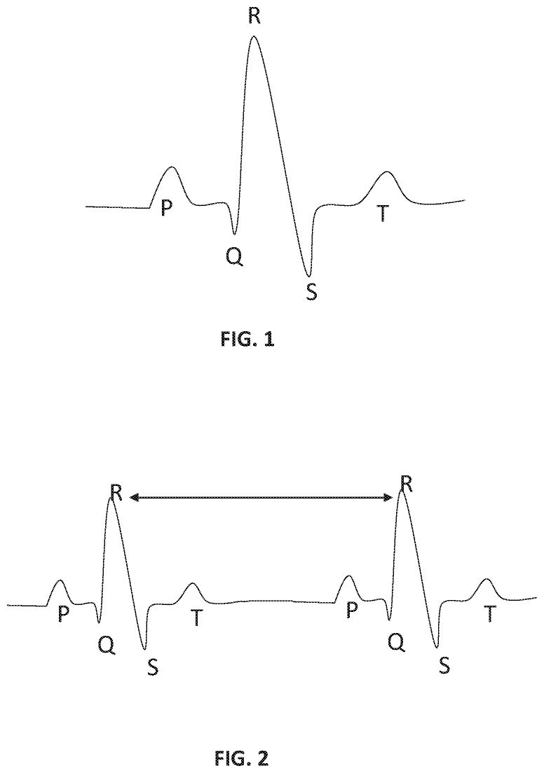

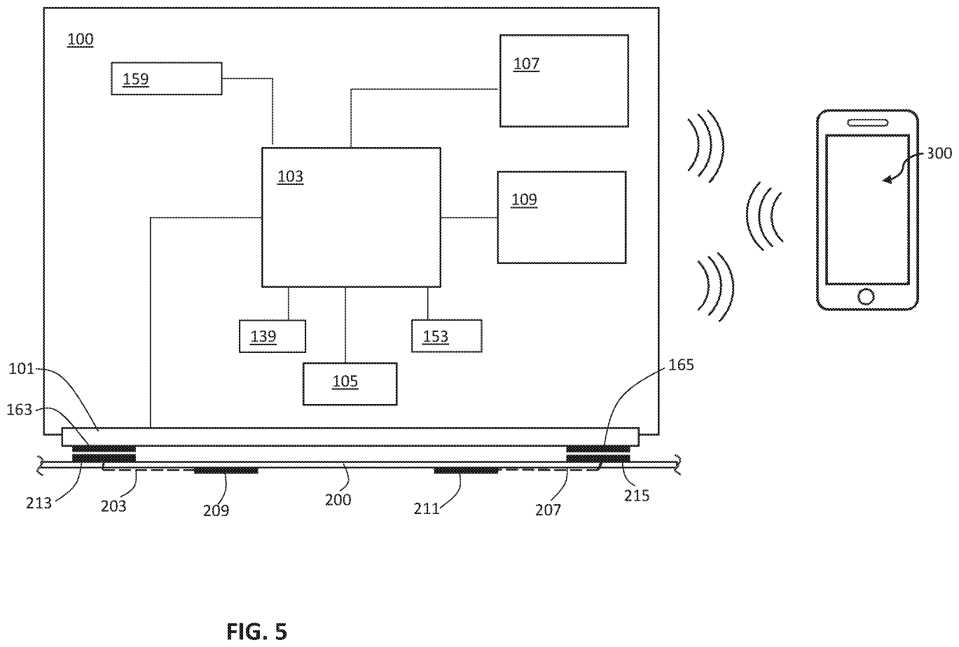

Referring to FIG. 5, there is shown a schematic diagram of an example of the electronics module 100 of FIG. 1.

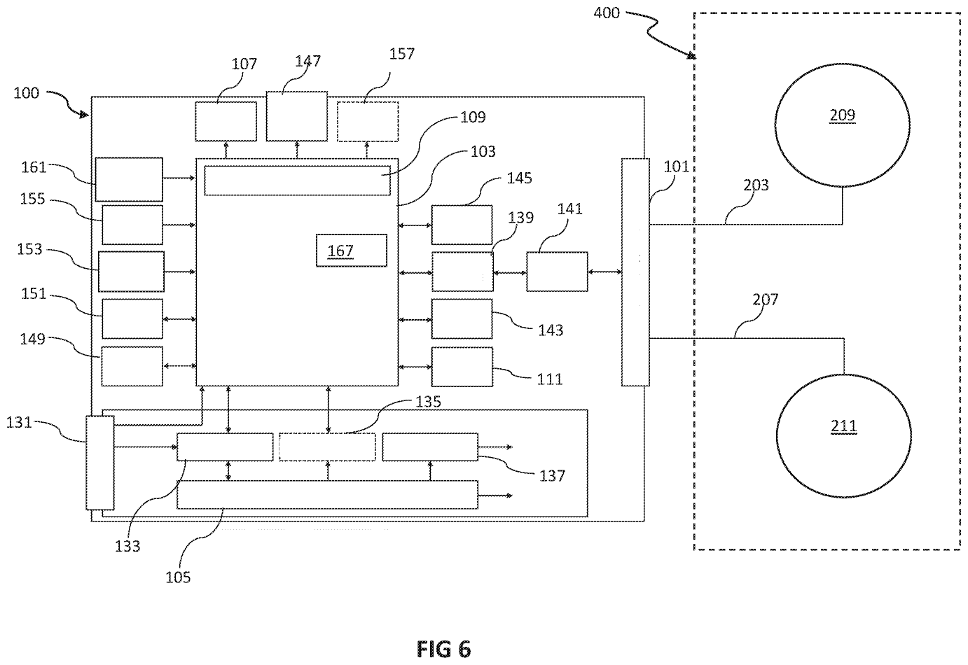

A more detailed block diagram of the electronics components of electronics module 100 and garment are shown in FIG. 6.

The electronics module 100 comprises an interface 101, a controller 103, a power source 105, and one or more communication devices which, in the exemplar embodiment comprises a first antenna 107, a second antenna 109 and a wireless communicator 159. The electronics module 100 also includes an input unit such as a proximity sensor or a motion sensor 111, for example in the form of an inertial measurement unit (IMU).

The electronics module 100 also includes additional peripheral devices that are used to perform specific functions as will be described in further detail herein.

The interface 101 is arranged to communicatively couple with the sensing unit 400 of the garment 200. The sensing unit 400 comprises—in this example—the two sensors 209, 211 coupled to respective first and second electrically conductive pathways 203, 207, each with respective termination points 213, 215. The interface 101 receives signals from the sensors 209, 211. The controller 103 is communicatively coupled to the interface 101 and is arranged to receive the signals from the interface 101 for further processing.

The interface 101 of the embodiment described herein comprises first and second contacts 163, 165 which are arranged to be communicatively coupled to the termination points 213, 215 the respective first and second electrically conductive pathways 203, 207. The coupling between the termination points 213, 215 and the respective first and second contacts 163, 165 may be conductive or a wireless (e.g. inductive) communication coupling.

In this example the sensors 209, 211 are used to measure electropotential signals such as electrocardiogram (ECG) signals, although the sensors 209, 211 could be configured to measure other biosignal types as also discussed above.

In this embodiment, the sensors 209, 211 are configured for so-called dry connection to the wearer's skin to measure ECG signals.

The power source 105 may comprise a plurality of power sources. The power source 105 may be a battery. The battery may be a rechargeable battery. The battery may be a rechargeable battery adapted to be charged wirelessly such as by inductive charging. The power source 105 may comprise an energy harvesting device. The energy harvesting device may be configured to generate electric power signals in response to kinetic events such as kinetic events 10 performed by the wearer 600 of the garment 200. The kinetic event could include walking, running, exercising or respiration of the wearer 600. The energy harvesting material may comprise a piezoelectric material which generates electricity in response to mechanical deformation of the converter. The energy harvesting device may harvest energy from body heat of the wearer 600 of the garment. The energy harvesting device may be a thermoelectric energy harvesting device. The power source 105 may be a super capacitor, or an energy cell.

The first antenna 107 is arranged to communicatively couple with the user electronic device 300 using a first communication protocol. In the example described herein the first antenna 107 is a passive tag such as a passive Radio Frequency Identification (RFID) tag or Near Field Communication (NFC) tag. These tags comprise a communication module as well as a memory which stores the information, and a radio chip. The user electronic device 300 is powered to induce a magnetic field in an antenna of the user electronic device 300. When the user electronic device 300 is placed in the magnetic field of the communication module antenna 107, the user electronic device 300 induces current in the communication module antenna 107. This induced current is used to retrieve the information from the memory of the tag and transmit the same back to the user electronic device 300. The controller 103 is arranged to energize the first antenna 107 to transmit information.

In an example operation, the user electronic device 300 is brought into proximity with the electronics module 100. In response to this, the electronics module 100 is configured to energize the first antenna 107 to transmit information to the user electronic device 300 over the first wireless communication protocol. Beneficially, this means that the act of the user electronic device 300 approaching the electronics module 100 energizes the first antenna 107 to transmit the information to the user electronic device 300.

The information may comprise a unique identifier for the electronics module 100. The unique identifier for the electronics module 100 may be an address for the electronics module 100 such as a MAC address or Bluetooth® address.

The information may comprise authentication information used to facilitate the pairing between the electronics module 100 and the user electronic device 300 over the second wireless communication protocol. This means that the transmitted information is used as part of an out of band (OOB) pairing process.

The information may comprise application information which may be used by the user electronic device 300 to start an application on the user electronic device 300 or configure an application running on the user electronic device 300. The application may be started on the user electronic device 300 automatically (e.g. without wearer 600 input). Alternatively, the application information may cause the user electronic device 300 to prompt the wearer 600 to start the application on the user electronic device. The information may comprise a uniform resource identifier such as a uniform resource location to be accessed by the user electronic device, or text to be displayed on the user electronic device for example. It will be appreciated that the same electronics module 100 can transmit any of the above example information either alone or in combination. The electronics module 100 may transmit different types of information depending on the current operational state of the electronics module 100 and based on information it receives from other devices such as the user electronic device 300.

The second antenna 109 is arranged to communicatively couple with the user electronic device 300 over a second wireless communication protocol. The second wireless communication protocol may be a Bluetooth® protocol, Bluetooth® 5 or a Bluetooth® Low Energy protocol but is not limited to any particular communication protocol. In the present embodiment, the second antenna 109 is integrated into controller 103. The second antenna 109 enables communication between the user electronic device 300 and the controller 100 for configuration and set up of the controller 103 and the peripheral devices as may be required. Configuration of the controller 103 and peripheral devices utilises the Bluetooth® protocol.

The wireless communicator 159 may be an alternative, or in addition to, the first and second antennas 107, 109.

Other wireless communication protocols can also be used, such as used for communication over: a wireless wide area network (WWAN), a wireless metro area network (WMAN), a wireless local area network (WLAN), a wireless personal area network (WPAN), Bluetooth® Low Energy, Bluetooth® Mesh, Thread, Zigbee, IEEE 802.15.4, Ant, a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The electronics module 100 includes configured a clock unit in the form of a real time clock (RTC) 153 coupled to the controller 103 and, for example, to be used for data logging, clock building, time stamping, timers, and alarms. As an example, the RTC 153 is driven by a low frequency clock source or crystal operated at 32.768 Hz.

The electronics module 100 also includes a location device 161 such as a GNSS (Global Navigation Satellite System) device which is arranged to provide location and position data for applications as required. In particular, the location device 161 provides geographical location data at least to a nation state level. Any device suitable for providing location, navigation or for tracking the position could be utilised. The GNSS device may include device may include Global Positioning System (GPS), BeiDou Navigation Satellite System (BDS) and the Galileo system devices.

The power source 105 in this example is a lithium polymer battery 105. The battery 105 is rechargeable and charged via a USB C input 131 of the electronics module 100. Of course, the present disclosure is not limited to recharging via USB and instead other forms of charging such as inductive of far field wireless charging are within the scope of the present disclosure. Additional battery management functionality is provided in terms of a charge controller 133, battery monitor 135 and regulator 147. These components may be provided through use of a 30 dedicated power management integrated circuit (PMIC).

The USB C input 131 is also coupled to the controller 131 to enable direct communication with the controller 103 with an external device if required.

The controller 103 is communicatively connected to a battery monitor 135 so that that the controller 103 may obtain information about the state of charge of the battery 105.

The controller 103 has an internal memory 167 and is also communicatively connected to an external memory 143 which in this example is a NAND Flash memory. The memory 143 is used to for the storage of data when no wireless connection is available between the electronics module 100 and a user electronic device 300. The memory 143 may have a storage capacity of at least 1 GB and preferably at least 2 GB.

The electronics module 100 also comprises a temperature sensor 145 and a light emitting diode 147 for conveying status information. The electronic module 100 also comprises conventional electronics components including a power-on-reset generator 149, a development connector 151, the real time clock 153 and a PROG header 155.

Additionally, the electronics module 100 may comprise a haptic feedback unit 157 for providing a haptic (vibrational) feedback to the wearer 600.

The wireless communicator 159 may provide wireless communication capabilities for the garment 200 and enables the garment to communicate via one or more wireless communication protocols to a remote server 700. Wireless communications may include: a wireless wide area network (WWAN), a wireless metro area network (WMAN), a wireless local area network (WLAN), a wireless personal area network (WPAN), Bluetooth® Low Energy, Bluetooth® Mesh, Bluetooth® 5, Thread, Zigbee, IEEE 802.15.4, Ant, a near field communication (NFC), a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The electronics module 100 may additionally comprise a Universal Integrated Circuit Card (UICC) that enables the garment to access services provided by a mobile network operator (MNO) or virtual mobile network operator (VMNO). The UICC may include at least a read-only memory (ROM) configured to store an MNO or VMNO profile that the garment can utilize to register and interact with an MNO or VMNO. The UICC may be in the form of a Subscriber Identity Module (SIM) card. The electronics module 100 may have a receiving section arranged to receive the SIM card. In other examples, the UICC is embedded directly into a controller of the electronics module 100. That is, the UICC may be an electronic/embedded UICC (eUICC). A eUICC is beneficial as it removes the need to store a number of MNO profiles, i.e. electronic Subscriber Identity Modules (eSIMs). Moreover, eSIMs can be remotely provisioned to garments. The electronics module 100 may comprise a secure element that represents an 35 embedded Universal Integrated Circuit Card (eUICC). In the present disclosure, the electronics module may also be referred to as an electronics device or unit. These terms may be used interchangeably.

The controller 103 is connected to the interface 101 via an analog-to-digital converter (ADC) front end 139 and an electrostatic discharge (ESD) protection circuit 141.

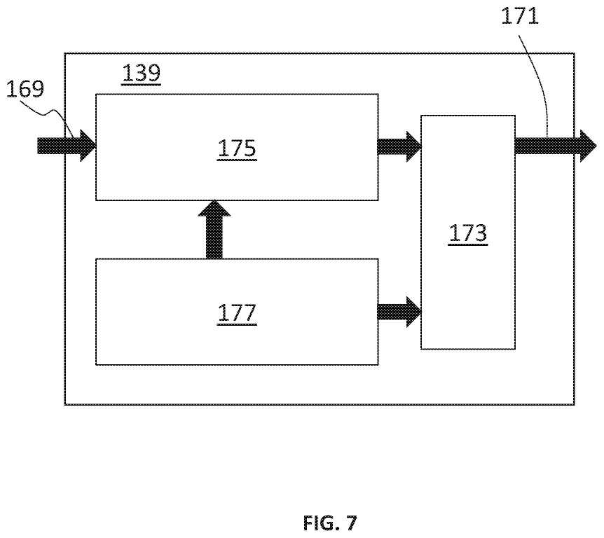

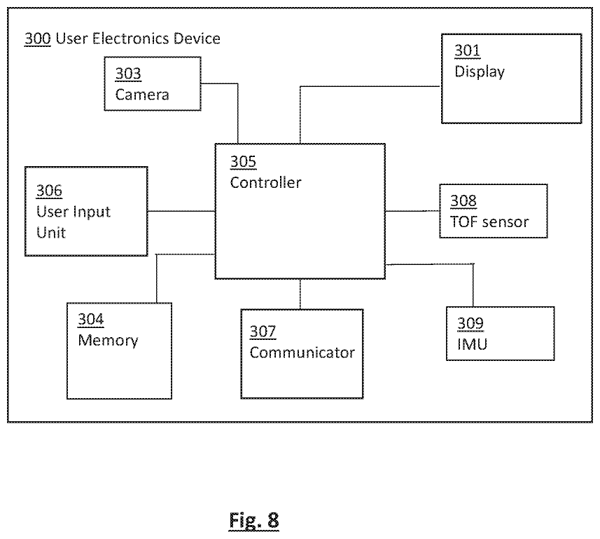

FIG. 7 is a schematic illustration of the component circuitry for the ADC front end 139.

In the example described herein the ADC front end 139 is an integrated circuit (IC) chip which converts the raw analogue biosignal received from the sensors 209, 211 into a digital signal for further processing by the controller 103. ADC IC chips are known, and any suitable one can be utilised to provide this functionality. ADC IC chips for ECG applications include, for example, the MAX30003 chip produced by Maxim Integrated Products Inc.

The ADC front end 139 includes an input 169 and an output 171.

Raw biosignals from the electrodes 209, 211 are input to the ADC front end 139, where received signals are processed in an ECG channel 175 and subject to appropriate filtering through high pass and low pass filters for static discharge and interference reduction as well as for reducing bandwidth prior to conversion to digital signals. The reduction in bandwidth is important to remove or reduce motion artefacts that give rise to noise in the signal due to movement of the sensors 209, 211 and the effect of impact forces on the electrical components.

The output digital signals may be decimated to reduce the sampling rate prior to being passed to a serial programmable interface (SPI) 173 of the ADC front end 139.

ADC front end IC chips suitable for ECG applications may be configured to extract and determine information from the input biosignals such as heart rate, IBI values and the QRS complex. Support circuitry 177 provides base voltages for the ECG channel 175.

The determining of the QRS complex, heart rate and IBI values can be done for example using the known Pan Tomkins algorithm as described in Pan, Jiapu; Tompkins, Willis J. (March 1985). “A Real-Time QRS Detection Algorithm”. IEEE Transactions on Biomedical Engineering. BME-32 (3): 230-236.

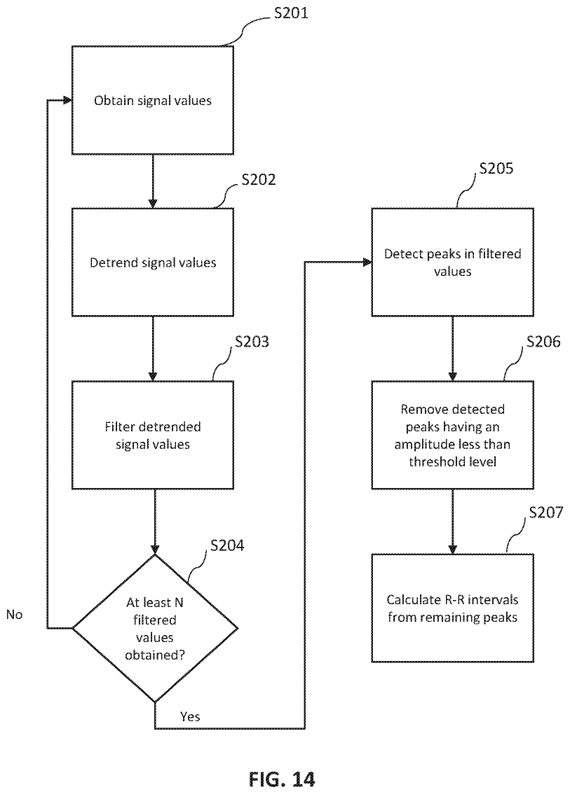

R peak detection and the IBI, for example as determined from the R-R interval can be determined on the controller 103, using, for example, the method detailed below with respect to FIG. 14.

Signals are output to the controller 103 via the SPI 173.

The controller 103 can also be configured to apply digital signal processing (DSP) to the digital signal from the ADC front end 139.

The DSP may include noise filtering additional to that carried out in the ADC front end 139 and may also include additional processing to determine further information about the signal from the ADC front end 139.

The digital signal values output to the controller 103 are stored in a FIFO data buffer. The controller 103 performs operations to calculate R-R intervals from the digital signal values. The operations are performed in real-time while the ADC front end 139 are outputting new digital signals to the controller 103. FIG. 14 provides a flow diagram for an example method performed by the controller 103 for calculating the IBI from the digital signals stored in the FIFO data buffer.

In step S201, the controller 103 reads signal values from the data buffer. Each of the signal values is a value that represents the amplitude of the ECG signal at a particular time point.

In step S202, the controller 103 detrends the signal values so as to remove baseline wander and/or other low frequency components. In an example operation, the controller 103 calculates the trend in the signal values and then subtracts the calculated trend from each of the signal values.

Calculating the trend comprises identifying the maximum and minimum signal values read from the data buffer. The maximum signal value is added to a buffer that stores the maximum signal values obtained over time. The minimum signal value is added to a buffer that stores the minimum signal values obtained over time. The current trend is then calculated by calculating the average of the maximum value stored in the buffer of maximum signal values and the minimum value stored in the buffer of minimum signal values.

As explained above, the detrended signal values are calculated by subtracting the calculated current trend from each of the signal values. The detrended signal values are added to a FIFO detrended signal buffer.

In step S203, the detrended signal values are filtered. The filtering is performed to remove components from the signal that do not resemble R-peaks. A bandpass filter centred around the frequency associated with the shape and width of the R-peak can be used to perform this task.

Some filtering approaches use a bandpass filter with a central frequency in the range of 17 to 19 Hz. IIR or FIR filters may be used, however, they are generally not effective due to ripples and lobes that may be present around the R peaks in the ECG signal. The interaction between these secondary peaks and other components of the ECG signal can lead to ambiguity in the identity of the actual main peak.

Preferred bandpass filtering approaches analyse a signal of the instantaneous amplitude associated with the R-peak frequency. These approaches exploit the fact that R-peaks are approximately symmetrical features which means that the location of the peak in the spectral amplitude is normally close to the location of the centre of the R-peak itself. The signal of instantaneous amplitude can be obtained using a complex filter and by calculating the absolute magnitude of the real and imaginary component for each filtered signal value.

In a preferred implementation, the complex filter used is a complex Morlet wavelet. The Morlet wavelet has optimal frequency resolution due to its Gaussian envelope. The Morlet wavelet is also useful because it is symmetrical across the y-axis which means that only half of the filter coefficients need to be stored in ram.

The filtered signal values are added to a FIFO filtered signal buffer.

In step S204, the controller 103 determines whether at least N filtered signal values have been obtained. Here, N is a number that may be selected by the skilled person as desired to ensure that there are likely to be a certain desired number of peaks within the window of filtered signal values. For example, N may be selected such that the filtered signal buffer contains at least 4 seconds of data to ensure that there are at least 2 peaks in any window. The number N will depend on the sampling rate of the signal values provided to the controller 103. For example, if the sampling rate is 512 Hz and at least 4 seconds of data are required, then N=2048. Other values of N are within the scope of the present disclosure.

If less than N samples of filtered signal values have been obtained then method returns to step S201 so that additional samples are gathered, filtered, and added to the filtered signal buffer. Steps S201 to S204 are repeated until the N signal values are obtained.

If N or more samples of the filtered signal values have been obtained, the method proceeds to step S205.

In step S205, the controller 103 detects peaks in the filtered signal values. At this stage, the controller 103 is identifying any peaks, including small and spurious peaks, in the filtered signal values. The peak detection process identifies local maxima in the signal values. Peak detection can be performed by simply looking for negative gradients in the filtered signal values.

In step S206, the controller 103 removes detected peaks that have an amplitude less than a threshold level. The thresholding process is intended to remove peaks that are not R-peaks in the ECG signal. The thresholding level is determined according to a configurable threshold multiplied by the average spectral power for the filtered signal values. Using the average spectral power enables the thresholding level to adapt based on the power of the signal.

In step S207, R-R intervals are calculated for the remaining. R-R intervals are calculated by calculating the difference between time stamps for consecutive R peaks. Only one R-R interval may be determined if only one R peak remains after step S206. The R-R interval will be determined using the timestamp of the last R-peak found in the previous window of data.

One or more additional steps may be performed prior to step S207 to such as to check the remaining peaks after step S206 and remove or compensate for spurious remaining peaks as will be described further below.

The ECG output, from the ADC front end 139 to the controller 103 is configured to measure time difference between the two R peaks in the ECG waveform. As discussed above, this is the inter-beat interval (IBI). The IBI will change depending upon whether a wearer is at rest or is in an active phase.

As also mentioned above, the ECG signals are sampled at a frequency of 512 Hz with a window size of 4 seconds which is equal to 2048 samples per window. Typically, this will give a minimum number of two peaks in a window: equivalent to a heart rate of 30 beats per minute, and a maximum number of 16 peaks equivalent to a heart rate of 240 beats per minute.

Detecting heart rate variability (HRV) is predicated on the understanding heart rate is incapable of rapid changes from its current value. If the HRV changes suddenly, this can be indicative of a spurious or missing heart beat or some other abnormal heart pattern.

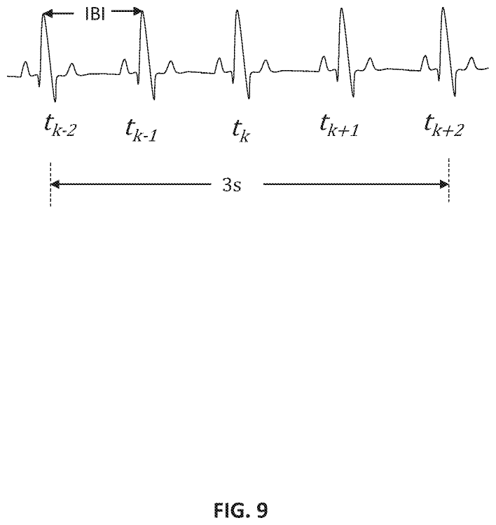

FIG. 9 illustrates a portion of heart rate trace. With five heartbeats in 3 seconds, the IBI is 0.75 seconds which corresponds to a heart rate, r, of 80 beats per minute.

In a normal heart rate pattern, the rate of change i.e. the derivative of heart rate rₖ′ with respect to time, must be below some threshold as given in (1):

$$\vert {r}_{k}^{′} \vert = 2 \vert \frac{{t}_{k - 1} - 2 {t}_{k} + {t}_{k + 1}}{( {t}_{k - 1} - {t}_{k} ) ( {t}_{k - 1} - {t}_{k + 1} ) ( {t}_{k} - {t}_{k + 1} )} \vert < U \qquad ( 1 )$$

Where:
rₖ′ is the derivative of the heart rate, at the k^{th} heart beat;
tₖ is the time of the k^{th} heart beat;
tₖ₋₁ and tₖ₊₁ are the times of the (k−1)^{th} and (k+1)^{th} heartbeat respectively, being either side of the k^{th} heartbeat; and
U is a predetermined configurable threshold value.

The configurable parameter, U, is used to set how fast a change should be treated as anomalous. If this condition is not met, then it means that peaks at tₖ or tₖ₊₁ are anomalous. The peak at tₖ₋₁ will have been checked in prior steps and corrected for if required. If an anomaly is detected, it can be corrected by one of the following steps:
1. Removing peak at tₖ
2. Removing peak at tₖ₊₁
3. Inserting a peak between peaks at positions tₖ₋₁ and tₖ
4. Inserting a peak between peaks at positions tₖ and tₖ₊₁
5. Moving the peak at tₖ to an intermediate position between tₖ₋₁ and tₖ₊₁
6. Move the peak at tₖ₊₁ to an intermediate position between tₖ and tₖ₊₂

The anomaly condition as defined by equation (1) above and the steps 1. to 6. are based on Mateo and Laguna: “Analysis of Heart Rate Variability in the Presence of Ectopic Beats Using the Heart Timing Signal” and referred to above.

The limitation of this method is that it is necessary to try different combinations of each of the six steps on multiple peaks in succession to work out the optimal correction. This requires a long chain of R peak values. A large chain is not available in a four second sampling window as in the present invention. It is also quite computationally expensive.

In view of this, the present invention uses equation (1) to give weighted ranking to each of the steps 1 to 6 to find the optimal fix for the anomaly.

An anomaly check of the present invention comprises two steps: firstly, for a k^{th} peak, the peak is assessed for any anomaly using equation (1) above. If an anomaly is detected because the rate of change of heart rate rₖ′ is determined to be above the predetermined threshold U, then, secondly, the steps 1 to 6 is carried out for that peak to find the optimal correction for the anomalous peak.

Because any anomaly correction for one peak i.e. by inserting, removing or moving a peak may change the IBI interval in relation to adjacent peaks, the adjacent peaks are checked to assess the impact of the anomaly correction. Thus, adjacent peaks are also anomaly checked. So, more than one anomaly check is performed for each anomaly correction step: a total of three—one on either side of the current peak at position k.

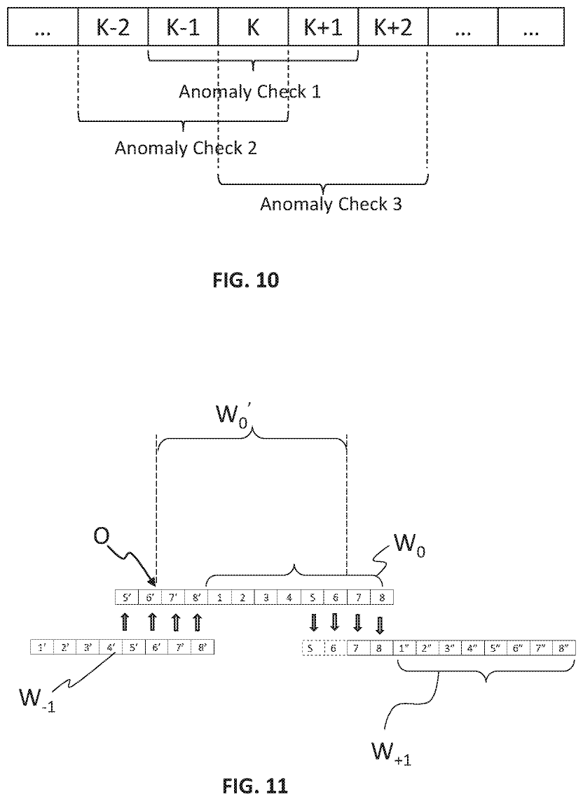

FIG. 10 illustrates this schematically. FIG. 10 shows illustrates a four second window W₀ sampling at 512 samples per second. As mentioned above, for any anomaly correction of a peak k, three anomaly checks are carried out; a first around the k^{th} peak, then a similar second anomaly check around the k−1^{th} peak, and then a third anomaly check around the k+1^{th} peak. In the example illustrated in FIG. 10, eight peaks are detected which is equivalent to a heart rate of 120 beats per minute.

The anomaly check is performed at each of the peaks in the sample window for all steps and saved to a buffer. The contribution from each anomaly check is summed, the total value is then used to decide on which anomaly correction step gave the lowest overall anomaly value.

As any k^{th} peak in a sampling window requires two peaks on either side of the current peak (see k−2, k−1; k+1, k+2 of FIG. 10) for the three-stage anomaly check to be carried out, it is evident that the last two peaks in any current window cannot be checked. The last two peak values in any window are therefore shifted in as the first two peaks in the next window. Similarly, because the two peak values that are shifted into the next window also require two previous values in order to carry out any anomaly checks on the shifted peak values, the previous two peaks are also needed. Thus, the last four peak values are brought forward.

In the present example, the term ‘value’ or ‘peak value’ is the time at which a peak is detected.

This is illustrated schematically in FIG. 11 which shows three windows W₋₁, W₀, W₊₁. The last two peaks of the previous window W₋₁ are moved into the current window W₀, whilst the last two peaks of the current window W₀ are stored in the buffer ready to be moved to the next window W₊₁. The time is referenced from the first peak of a window that is stored in the buffer. In FIG. 11, for example, the current window W₀ is stored in the buffer and the timing of each detected peak is with reference to the peak 5′ i.e. the first peak in the buffer. As discussed above, two peaks need to be available to validate these last two peaks of the previous window so, in total, the last four peaks are stored in the buffer and then moved into the current window.

In the example illustrated in FIG. 11, a current sample window W₀ includes eight peaks: peaks 1 through to peak 8. However, with the current method, peaks 7 and 8 cannot be checked in the current window W₀, so peaks 5, 6, 7 and 8 are stored in a buffer for use in the next window W₊₁. In the previous window W₋₁, peaks 5′ to 8′ will have been similarly stored and are then shifted to the current window W₀. Peaks 7′ and 8′ will be checked as part of the current window W₀, with peaks 5′ and 6′ used for validation in the steps as described below but are passed as checked from the current window W₀. Similarly, peaks 5 to 8 of the current window W₀ will be shifted into the next window W₊₁ with peaks 7 and 8 being checked as part of that window and peaks 5 and 6 being used in validation.

The origin, O, of the current window W₀ is shifted to ensure that the correct unchecked peaks are checked. FIG. 11 shows how the current window of peaks being checked is indicated at W₀′.

When the system 10 starts up, the electronics module 100 starts to receive biosignal data from the sensors 400, which is processed by the ADC front end 139 and sent to the controller 103. At start up, the first window will not be checked, but the last four peaks will be passed to the next window for checking.

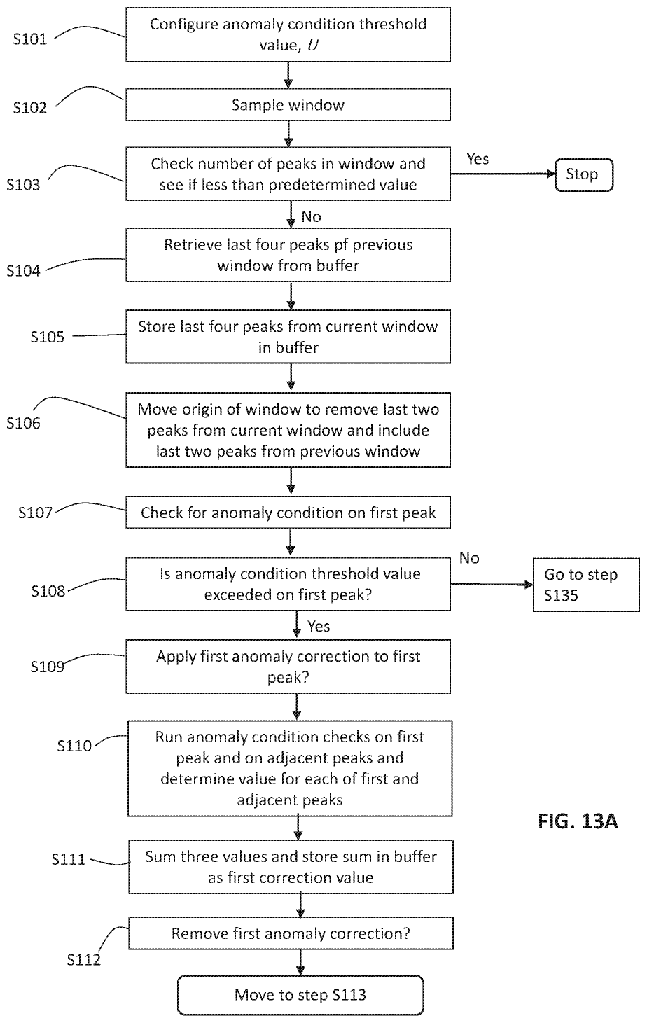

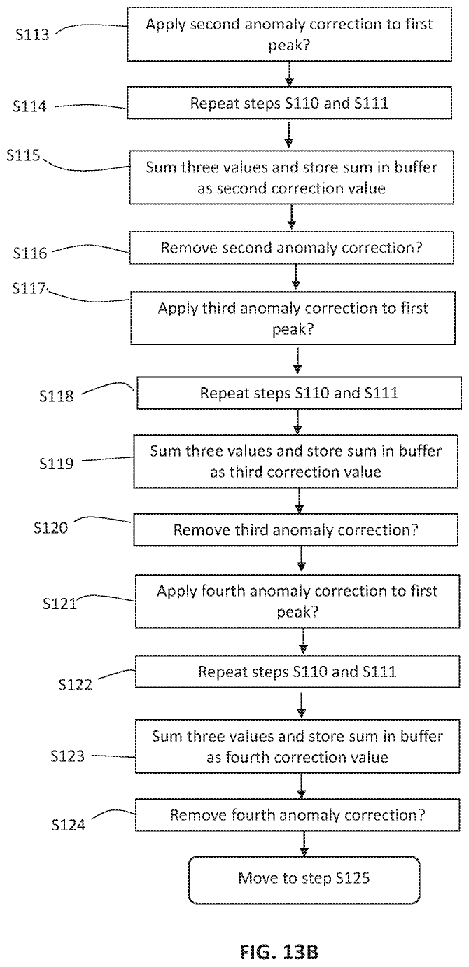

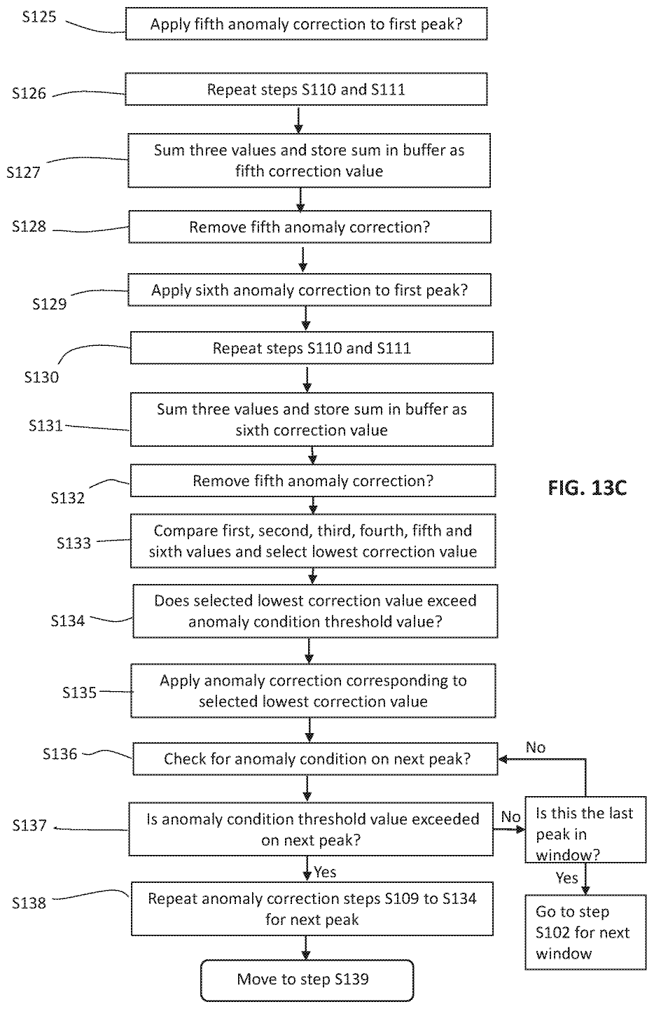

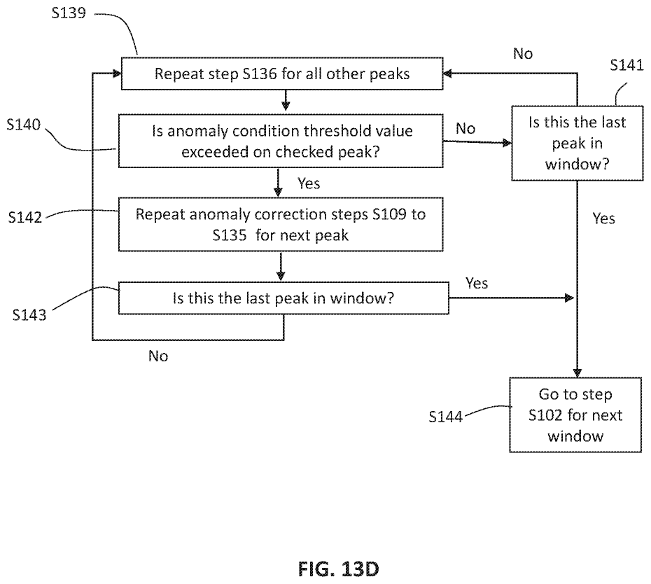

An example of the computer-implemented method of the present invention is described with respect to FIGS. 13A to 13D. In the present embodiment, the method of the present invention is implemented on the controller 103, although this method could be implemented on other devices.

In step S101, the anomaly condition threshold value, U is configured. In the present example, U is set at 1×10⁻⁶.

As described above, at step S102, the controller 103 is configured to sample the ECG data in four second windows W₋₁, W₀. W₊₁. Sampling is done at 512 samples per second i.e. a total of 2048 samples in window. In any window, a number of heart beats will be detected. In an example described herein, consider that eight heart beats are sampled in a four second window which is around 120 beats per minute. In a normal ECG trace, the inter-beat interval will be relatively stable and the rate of change of heart rate will be around or close to zero when the wearer 600 is stationary.

For each window, at step S103, the number of peaks is counted and if there are less than a predetermined number then the operation is stopped. In the example described herein, the predetermined number is four. With any less than four peaks in a window, the biosignal data is considered meaningless.

Otherwise, the next step S104 is for the last four peaks from the previous window, and that have been stored in a buffer, are retrieved and stored with the peaks of the current window.

At step S105, the last four peaks of the current window are stored in a buffer and then at step S106, the origin of the current window is shifted to include the last two peaks of the previous window and to remove the last two of the current window. This can be seen in FIG. 11 which shows the origin, O, moved so that the current window W₀ includes peaks 7′ and 8′ of the previous window W₋₁, but excludes peaks 7 and 8, which will be checked in the next window W₊₁. The peaks that will be checked are indicated by W₀ in FIG. 11.

The next part of the method is to check the peaks in the current window to check whether there is any anomaly present and, if so, to proceed through correction steps and to determine the most appropriate correction, as will be described further below.

The first peak—7′ in FIG. 11—is anomaly checked at step S107 using equation (1). In the example of FIG. 11, for the first peak 7′ the values used in equation (1) are the timings of peaks 6′ and 8′.

If the anomaly condition threshold value is less than the predetermined value U, then the process moves on to the next peak, 8′, because no anomaly has been identified.

Otherwise, the next steps are to sequentially apply the anomaly correction steps 1 to 6 as described further and test using equation (1).

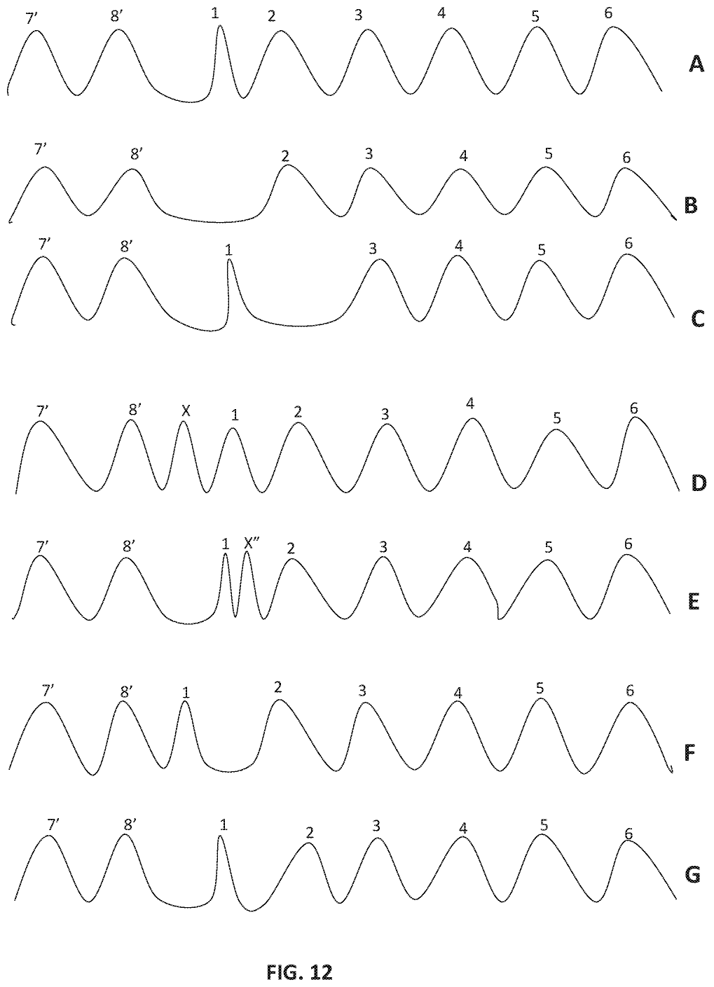

This is best described with reference to the example of FIGS. 11 and 12.

FIG. 12 illustrates a schematic eight-peak window in which the different anomaly correction steps are applied.

Trace A is a sampled window without correction. Each peak is labelled in correspondence to FIG. 11, so the first peak in the window is peak 7′ and the last peak 6.

Consider that peaks 7′ and 8′ are identified as normal as the anomaly condition threshold value is less than the predetermined value, U in both instances.

However, peak 1 is determined to be anomalous because equation (1) when applied to peak 1 exceeds the anomaly condition threshold value U.

The first anomaly correction will then be applied to peak 1 at step S109. This first anomaly correction comprises removing peak 1 (see trace B of FIG. 12).

At step S110, anomaly condition checks are carried out on peak 2 (peak 1 having being removed and peak 2 being the nearest) and the two adjacent peaks 3 and 8′ either side of peak 2. The values of the anomaly condition threshold value U for each of the three peaks 8′, 2, 3 are determined and, at step S111, these values are summed to generate a first correction value which is stored in a buffer. The first anomaly correction is then removed at step S112 by restoring peak 1 which had been removed.

The second anomaly correction will then be applied to peak 1 at step S113. This second anomaly correction comprises removing peak 2 (see trace C of FIG. 12).

At step S114, anomaly condition checks are carried out on peak 3 (peak 2 having being removed and peak 3 being the nearest) and the two adjacent peaks 1 and 4 either side of peak 3. The values of the anomaly condition threshold value U for each of the three peaks 1, 3, 4 are determined and then summed (at step S115) to generate a second correction value which is stored in a buffer. The second anomaly correction is then removed at step S116 by restoring peak 2 which had been removed.

The third anomaly correction will then be applied to peak 1 at step S117. This third anomaly correction comprises inserting a peak between peaks 8′ and 1 (see trace D of FIG. 12 which shows an extra peak X).

At step S118, anomaly condition checks are carried out on peak 1 and the two adjacent peaks X and 2 which are either side of peak 1. The values of the anomaly condition threshold value U for each of the three peaks X, 1, 2 are determined and summed (in step S119) to generate a third correction value which is stored in a buffer. The third anomaly correction is then removed at step S120 by removing the peak X which had been inserted.

The fourth anomaly correction will then be applied to peak 1 at step S121. This fourth anomaly correction comprises inserting a peak between peaks 1 and 2 (see trace E of FIG. 12 which shows an extra peak X″).

At step S122, anomaly condition checks are carried out on peak 1 and the two adjacent peaks 8′ and X″ which are either side of peak 1. The values of the anomaly condition threshold value U for each of the three peaks 8′, 1 and X″ are determined and then summed (in step S123) to generate a fourth correction value which is stored in a buffer. The fourth anomaly correction is then removed at step S124 by removing the peak X″ which had been inserted.

The fifth anomaly correction will then be applied to peak 1 at step S125. This fifth anomaly correction comprises moving peak 1 to an intermediate position between peak 8′ and peak 2 (see trace F of FIG. 12).

At step S126, anomaly condition checks are carried out on peak 1 and the two adjacent peaks 8′ and 2 which are either side of peak 1. The values of the anomaly condition threshold value U for each of the three peaks 8′, 1 and 2 are determined and summed (in step S127) to generate a fifth correction value which is stored in a buffer. The fifth anomaly correction is then removed at step S128 by returning peak 1 to its original position.

The sixth anomaly correction will then be applied to peak 1 at step S129. This sixth anomaly correction comprises moving peak 1 to an intermediate position between peak 1 and peak 3 (see trace G of FIG. 12).

At step S130, anomaly condition checks are carried out on peak 1 and the two adjacent peaks 8′ and 2 which are either side of peak 1. The values of the anomaly condition threshold value U for each of the three peaks 8′, 1 and 2 are determined and then summed (in step S131) to generate a fifth correction value which is stored in a buffer. The sixth anomaly correction is then removed at step S132 by returning peak 1 to its original position.

Once the sixth anomaly correction check has been completed, at step S133 the first, second, third, fourth, fifth and sixth anomaly correction values are compared, and the lowest correction value is selected and this lowest value analysed—at step S134—to see if it less than the anomaly condition threshold value U.

If the value of the anomaly condition threshold value U is below the predetermined value, then the applied anomaly correction from which this value derived is applied permanently—at step S135. As an example, if the lowest anomaly correction value is the third correction value, this corresponds to the third anomaly correction in which a peak is inserted between the peaks adjacent either side of the peak deemed anomalous (see trace D in FIG. 12).

If none of the steps provide a summed value which is less than the predetermined value, then none of the anomaly corrections are applied and the anomaly is left. This is to avoid over correction or a cascading of incorrect values.

The method then proceeds, at step S137, to check the next peak in the current window. If this peak is deemed non-anomalous, then the method looks at the subsequent peak and so on.

If any peak is deemed to be anomalous, then the method moves to repeat steps S109 to S135 for that anomalous peak.

When the last peak in the current window has been checked, then the method moves on to the next window in accordance with step S102 and proceeds to check the peaks in the next, now current, window in the same way as described above.

In this way, anomalous heart beats and heart rate variations can be identified and corrected such that the ECG data is more informed to provide better heart rate insight.

The controller 103 is configured to send the biosignals to the user electronic device 300 using either of the first antenna 107, second antenna 109, or wireless communicator 159.

In some examples, the input unit—such as a proximity sensor or motion sensor—is arranged to detect a displacement of the electronics module 100. These displacements of the electronics module 100 may be caused by the object being tapped against the electronics module 100 or by the wearer 600 of the electronics module 100 being in motion, for example walking or running, or simply getting up from a recumbent position.

In the exemplar embodiment described herein, motion detection is provided by the IMU 111 which may comprise an accelerometer and optionally one or both of a gyroscope and a magnetometer. A gyroscope/magnetometer is not required in all examples, and instead only an accelerometer may be provided, or a gyroscope/magnetometer may be present but put into a low power state.

The input event could be provided by artificial intelligence (AI) and, as such, the input unit could be an AI system, machine or engine.

The IMU 111 can therefore be used to detect orientation and gestures with event-detection interrupts enabling motion tracking and contextual awareness. It has recognition of free-fall events, tap and double-tap sensing, activity or inactivity, stationary/motion detection, and wakeup events in addition to six dimensional orientation. A single tap, for example, can be used enable toggling through various modes or waking the electronics module 100 from a low power mode.

Known examples of IMUs that can be used for this application include the ST LSM6DSOX manufactured by STMicroelectronics. This IMU a system-in-package IMU featuring a 3D digital accelerometer and a 3D digital gyroscope.

Another example of a known IMU suitable for this application is the LSM6DSO also be STMicroelectronics.

The IMU 111 can include machine learning functionality, for example as provided in the ST LSM6DSOX. The machine learning functionality is implemented in a machine learning core (MLC).

The machine earning processing capability uses decision-tree logic. The MLC is an embedded feature of the IMU 111 and comprises a set of configurable parameters and decision trees.

As is understood in the art, decision tree is a mathematical tool composed of a series of configurable nodes. Each node is characterized by an “if-then-else” condition, where an input signal (represented by statistical parameters calculated from the sensor data) is evaluated against a threshold.

Decision trees are stored and generate results in the dedicated output registers. The results of the decision tree can be read from the application processor at any time. Furthermore, there is the possibility to generate an interrupt for every change in the result in the decision tree, which is beneficial in maintaining low-power consumption.

Decision trees can be generated using known machine learning tool such as Weka developed by the University of Waikato or using MATLAB or Python. In an example operation, the wearer 600 has positioned the electronics module 100 within the pocket 201 (FIG. 1) of the garment 200 and is wearing the garment 200. The wearer 600 taps their hand or mobile phone 300 against the pocket 201 and this tap event is detected by the input unit, which in this exemplar embodiment is the IMU 111. The IMU 111 sends a signal to the controller 103 to wake-up the controller 103 from the low power mode.

A processor of the IMU 111 may perform processing tasks to classify different types of detected motion. The processor of the IMU 111 may use the machine-learning functions so as to perform this classification. Performing the processing operations on the IMU 111 rather than the controller 103 is beneficial as it reduces power consumption and leaves the controller 103 free to perform other tasks. In addition, it allows for motion events to be detected even when the controller 103 is operating in a low power mode.

The IMU 111 may be configured to detect when the electronic device 100 has been stationary but then begins to move, for example when left on a surface but then attached to the garment 200. The IMU 111 may be configured to detect that the wearer 600 of the garment 200, with the electronic device attached, is resting, or is moving, for example during exercise.

The IMU 111 communicates with the controller 103 over a serial protocol such as the Serial Peripheral Interface (SPI), Inter-Integrated Circuit (I2C), Controller Area Network (CAN), and Recommended Standard 232 (RS-232). Other serial protocols are within the scope of the present disclosure. The IMU 111 is also able to send interrupt signals to the controller 103 when required so as to transition the controller 103 from a low power model to a normal power mode when a motion event is detected, for example, or vice versa. The interrupt signals may be transmitted via one or more dedicated interrupt pins.

The user electronic device 300 in the example of FIGS. 3 and 7 is in the form of a mobile phone or tablet and comprises a controller 305, a memory 304, a wireless communicator 307, a display 301, a user input unit 306, a capturing device in the form of a camera 303 and an inertial measurement unit (IMU) 309. The controller 305 provides overall control to the user electronic device 300.

The user input unit 306 receives inputs from the user such as a user credential.

The memory 304 stores information for the user electronic device 300.

The display 301 is arranged to display a user interface 302 for applications operable on the user electronic device 300.

The IMU 309 provides motion and/or orientation detection and may comprise an accelerometer and optionally one or both of a gyroscope and a magnetometer.

The user electronic device 300 may also include a biometric sensor. The biometric sensor may be used to identify a user or users of device based on unique physiological features. The biometric sensor may be: a fingerprint sensor used to capture an image of a user's fingerprint; an iris scanner or a retina scanner configured to capture an image of a user's iris or retina; an ECG module used to measure the user's ECG; or the camera of the user electronic arranged to capture the face of the user. The biometric sensor may be an internal module of the user electronic device. The biometric module may be an external (stand-alone) device which may be coupled to the user electronic device by a wired or wireless link.

The controller 305 is configured to launch an application which is configured to display insights derived from the biosignal data processed by the ADC front end 139 of the electronics module 100, input to electronics module controller 103, and then transmitted from the electronics module 100. The transmitted data is received by the wireless communicator 307 of the user electronic device 300 and input to the controller 305.

Insights include, but are not limited to, an ECG signal trace i.e. the QRS complex, heart rate, respiration rate, core temperature but can also include identification data for the wearer 600 using the wearable assembly 500.

The display 301 is configured to display the ECG signal trace as part of the user interface 302, for example the ECG signal trace 800 as illustrated in FIG. 3. In the case of an ECG signal trace, the trace displayed is that corrected using the anomaly correction functionality described above. Other insights and data can be displayed on the display 301 as part of the user interface 302 as required.

The ECG trace 800 can be displayed in real-time.

The ECG trace 800 provides the user e.g. the wearer 600, with a visual representation of the wearer's heart's QRS complex including the IBI

The display 301 may be a presence-sensitive display and therefore may comprise the user input unit 306. The presence-sensitive display may include a display component and a presence-sensitive input component. The presence sensitive display may be a touch-screen display arranged as part of the user interface 302.

User electronic devices in accordance with the present invention are not limited to mobile phones or tablets and may take the form of any electronic device which may be used by a user to perform the methods according to aspects of the present invention. The user electronic device 300 may be a electronics module such as a smartphone, tablet personal computer (PC), mobile phone, smart phone, video telephone, laptop PC, netbook computer, personal digital assistant (PDA), mobile medical device, camera or wearable device. The user electronic device 300 may include a head-mounted device such as an Augmented Reality, Virtual Reality or Mixed Reality head-mounted device. The user electronic device 300 may be desktop PC, workstations, television apparatus or a projector, e.g. arranged to project a display onto a surface.

As described above, in use, the electronics module 100 is configured to receive raw biosignal data from the sensors 209, 211 and which are coupled to the controller 103 via the interface 101 and the ADC front end 139 for further processing and transmission to the user electronic device 300. The data transmitted to the user electronics device 300 includes raw and/or processed biosignal data such as ECG data, heart rate, respiration data, core temperature and other insights as determined.

The controller 305 of the user electronics device 300 is configured to receive, process and display data, such as the raw or processed biosignal data from the electronics module 100, for example via the application launched on the user electronics device 300. The wearer 600, is able to configure the application, using user inputs, to receive, process and display the received data in accordance with these user inputs.

The user electronic device 300 is arranged to receive the transmitted data from the electronics module 100 via the communicator 307 and which are coupled to the controller 305, and then to process and display the data in accordance with the user configuration.

The system 10 can be configured to detect when the wearer 600 is engaged in activity and display an ECG trace 800 along with additional data such as heart rate which is more easily viewed in these circumstances.

As mentioned above, the IMU 111 of the electronics module 100 can be configured to use decision tree logic to determine the activity level of the wearer of the electronics module 100 and to provide an output to the controller 103.

The controller 103 is also configured to determine (and transmit, for example via the second antenna 109 using Bluetooth® if required), activity classification data relating to this activity level, under an Activity Characteristic, for example 0=lying down, 1=sitting, 2=walking, 3=jogging, and 4=running.

As an alternative to providing activity classification date using machine learning, the raw data from the IMU 111 can be used by the electronics module 100, user electronic device 300 or a remote server 700 to perform activity classification.

The user electronic device 300 can also use motion data from the IMU 309 provided within the user electronic device 300 to determine activity levels of a user.

The IMU 139 on the user electronic device 300 can be used to validate activity events that are transmitted from the electronics module 100.

Optionally, the controller 305 of the user electronic device 300 can be configured to carry out image processing of the incoming ECG waveform to determine the quality of the biosignal data i.e. how noisy the ECG trace will be.

Alternatively, image processing can be implemented on a remote server 700 to which the electronics module 100 and user electronics device 300 are communicatively coupled.

The activity that is identified by the IMU 111 can be used by the controller 103 to determine which R peak detection process to use: that is the Pan-Tomkins algorithm on the ADC front end 139 or the more complex process on the controller 103 and described with reference to FIG. 14. If the IMU 111 identifies that the wearer 600 is laying down, sitting down, standing or walking then the microcontroller will simply use the IBI as determined from the R-R interval information that comes directly from the ADC front end 139. If, however, the IMU 111 identifies that the wearer 600 is jogging or running then the controller 103 can utilise the process on the controller 103 to obtain the IBI.

The R peak detection process performed on the ADC front end 139 is more susceptible to the pace noise as well as artefacts from other sources. The R peak detection functionality embedded in the controller 103 can remove artefacts that may otherwise cause incorrect R peak detection. This is far better suited for high activity scenarios.

During low activity, the aforementioned artefacts will not be present, and will be unlikely to cause inaccurate R peak detection when using the ADC front end 139. In these cases, it would be beneficial to use the ADC front end 139, since it draws far less power than the microcontroller without a loss in accuracy. It's also worth noting that low activity is where the wearer 600 will spend most of their time, and so we could stand to gain a large battery life improvement.

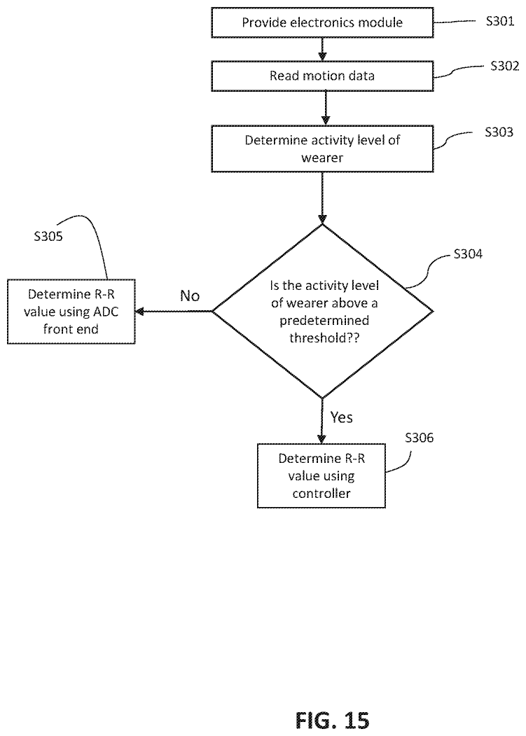

FIG. 15 provides a flow diagram for an example method performed by the controller 103 for selecting an R-R detection method.

In step S301 an electronics module is provided.

At step S302 motion data is read from the IMU 111 on the electronics module 100.

At step S303, the controller 103 (or IMU 111) is operable to determine the activity level of the wearer 600 as described above. For example, the activity level can be characterised by an Activity Characteristic, for example 0=lying down, 1=sitting, 2=walking, 3=jogging, and 4=running.

At step S304, the activity level is assessed as to whether it is above or below a predetermined threshold level. As an example, this could be whether the activity level is 0 or 1, or whether it is 2 or above.

If the activity level is below the predetermined threshold level, then, at step S305, the controller 103 is operable to read the R-R interval values from the ADC front end 139.

However, if the activity level is above the predetermined threshold level, then, at step S306, the controller 103 is operable to determine the R-R interval values.

In an alternative the motion data could be read from the user electronics device 300 using the IMU 309. The controller 305 could then be configured to determine the activity level of the wearer 600 and transmit this activity level the communicator 307, for example using Bluetooth®, to electronics module 100.

As mentioned above, the R peak detection functionality embedded in the controller 103 can remove artefacts that may otherwise cause incorrect R peak detection. This is far better suited for high activity scenarios.

During low activity, noise artefacts will not be present, and will be unlikely to cause inaccurate R peak detection.

In another example of the present invention, the controller 103 obtains subject specific information indicative of the likely signal-to-noise ratio of the ECG signal and this can be used to configure the threshold value, U, of equation (1).

The subject specific information provides further context to the ECG signal values and is used to configure the threshold value, U, of equation (1). The subject specific information may comprise information relating to the activity of the subject and/or a characteristic of the subject.

The subject specific information is indicative of a likely signal-to-noise ratio for the ECG signal. If the subject is likely to have a high signal-to-noise ratio for their ECG signal then a high threshold value, U, can be selected because the true R-peaks in the ECG signal are likely to have a far higher amplitude than spurious peaks caused by noise, for example. In this instance, the value of U is selected to be a value that is known to be significantly higher than any possible rate of change of heart rate where rₖ′.

If the subject is likely to have a low signal-to-noise ratio then a low threshold value, U, can be selected because the true R-peaks in the signal are likely to have a lower amplitude and may be missed if the threshold level is too high. The present disclosure therefore advantageously configures the threshold value U, according to the likely signal-to-noise ratio for the ECG signal as identified by the subject specific information.

The signal-to-noise ratio is not calculated directly from the ECG signal. Instead, the subject specific information provides information relating to one or more properties of the subject that indicate a likely signal-to-noise ratio. This is computationally more efficient than calculating the signal-to-noise ratio directly and allows for real time R-peak detection and removal even at a high sampling rate such as 512 Hz.

In some examples, the subject specific information for the subject comprises activity data for the subject such as an activity level of the subject. If the user has a high activity level (such as when they are running), the signal-to-noise ratio for the ECG signal is likely to be lower due to the introduction of greater motion artefacts. By setting the threshold value U, to compensate for the decreased signal-to-noise ratio, the controller 103 is able to reduce the number of missed true R-peaks. Meanwhile, if the user has a low activity level (such as when they are sitting or standing), the signal-to-noise ratio for the ECG signal is likely to be higher. By adjusting the threshold level to compensate for the increased signal-to-noise ratio, the controller 103 is able to remove a greater number of spurious peaks.

In some examples, the subject specific information for the subject comprises information indicating a characteristic of the subject or person whose electrocardiogram output is being monitored. Different characteristics of the user can be indicative of different signal-to-noise ratios that will be expected in the ECG signal. Factors such as gender, weight, and body fat level are indicative of different signal-to-noise ratios in the ECG signals for the subject. The controller 103 is able to set the threshold value U based on the particular characteristics of the subject to provide subject-specific R-peak detection.

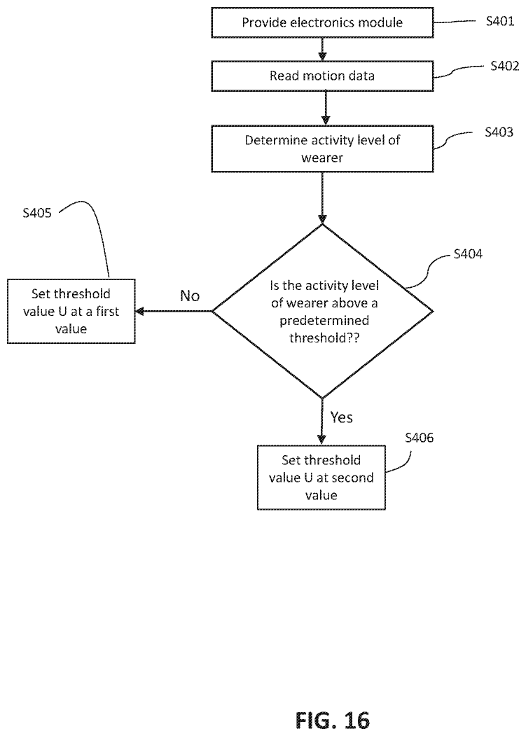

FIG. 16 provides a flow diagram for an example method performed by the controller 103 for selecting an R-R detection method.

In step S401 an electronics module is provided.

At step S402 motion data is read from the IMU 111 on the electronics module 100.

At step S403, the controller 103 (or IMU 111) is operable to determine the activity level of the wearer 600 as described above. For example, the activity level can be characterised by an Activity Characteristic, for example 0=lying down, 1=sitting, 2=walking, 3=jogging, and 4=running.

At step S404, the activity level is assessed as to whether it is above or below a predetermined threshold level. As an example, this could be whether the activity level is 0 or 1, or whether it is 2 or above.

If the activity level is below the predetermined threshold level, then, at step S405, the controller 103 is operable to set the threshold value U at a first value.

However, if the activity level is above the predetermined threshold level, then, at step S406, the controller 103 is operable to set the threshold value U at second value.

The first and second values can be determined using any suitable empirical determination.

In an alternative the motion data could be read from the user electronics device 300 using the IMU 309. The controller 305 could then be configured to determine the activity level of the wearer 600 and transmit this activity level the communicator 307, for example using Bluetooth®, to electronics module 100.

Whilst the steps in the example embodiments described above are implemented on specific components of the system 10, it will be understood that other combinations are possible. For example, steps implemented on the wearable assembly 500 user electronic device 300 or server 700 could equally be carried out on another of the wearable assembly 500, user electronic device or server.

In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A computer-implemented method of correcting an electrocardiogram output for anomalous heart beats, the method comprising:
obtaining a series heartrate values for the electrocardiogram output;
determining when the rate of change of the heartrate values exceeds a predetermined threshold value; and, in response to the predetermined threshold value being exceeded, sequentially applying one or more correcting steps to a region of the electrocardiogram output around a selected heartbeat where the rate of change of heartrate values occurred;
obtaining information indicative of the likely signal-to-noise ratio of the electrocardiogram output;
configuring the predetermined threshold value based on the likely signal-to-noise ratio;
determining the rate of change of heartrate values at the selected region for each of the applied correcting steps; and
choosing a correcting step corresponding to the lowest of the rate of change of heartrate values at the selected region as a permanent correction for the electrocardiogram output.

2. The computer-implemented method according to claim 1, wherein the predetermined threshold value is configured to be a first value when the likely signal-to-noise ratio is above a first predetermined value.

3. The computer-implemented method according to claim 1, wherein the predetermined threshold value is configured to be a second value when the likely signal-to-noise ratio is above a second predetermined value.

4. The computer-implemented method according to claim 1, wherein the obtained information relates to the activity level of a person whose electrocardiogram output is being monitored.

5. The computer-implemented method according to claim 1, wherein the obtained information relates to a characteristic of a person whose electrocardiogram output is being monitored.

6. (canceled)

7. The computer-implemented method according to claim 1, wherein the rate of change of heart rate is derived from the equation:
$$\vert {r}_{k}^{′} \vert = 2 \vert \frac{{t}_{k - 1} - 2 {t}_{k} + {t}_{k + 1}}{( {t}_{k - 1} - {t}_{k} ) ( {t}_{k - 1} - {t}_{k + 1} ) ( {t}_{k} - {t}_{k + 1} )} \vert$$
where rₖ′ is the rate of change of the heart rate, at the k^{th} heartbeat,
tₖ is the time of the k^{th} heartbeat, and
tₖ₋₁ and tₖ₊₁ are the times of the (k−1)^{th} and (k+1)^{th} heartbeat respectively either side of the k^{th} heartbeat.

8. The computer-implemented method according to claim 1, wherein the step of obtaining a series heartrate values for the electrocardiogram comprises a step of sampling a series of heart beats in the electrocardiogram output and determining the heart rate values from the sampled heart beats.

9. The computer-implemented method according to claim 8, in which the step of obtaining a series heartrate values for the electrocardiogram comprises sampling a first set of heart beats and a second set of heart beats, removing the last two heart beats from the first set of heart beats, removing the last two heart beats of the second set of heart beats and combining the last two heart beats from the first set of heart beats with the remaining heart beats from the second set of heart beats.

10. The computer-readable medium having instructions recorded thereon which, when executed by a processor, cause the processor to perform the method of claim 1.

11. A system for correcting an electrocardiogram output for anomalous heart beats, the system comprising a controller and a memory, the memory storing instructions which when executed by the controller cause the controller to perform operations comprising:
obtaining a series heartrate values for the electrocardiogram output;
determining when the rate of change of the heartrate values exceeds a predetermined threshold value; and, in response to the predetermined threshold value being exceeded,
sequentially applying one or more correcting steps to a region of the electrocardiogram output around a selected heartbeat where the rate of change of heartrate values occurred;
obtaining information indicative of the likely signal-to-noise ratio of the electrocardiogram output;
configuring the predetermined threshold value based on the likely signal-to-noise ratio; and
determining the rate of change of heartrate values at the selected region for each of the applied correcting steps; and
choosing a correcting step corresponding to the lowest of the rate of change of heartrate values at the selected region as a permanent correction for the electrocardiogram output.

12. The system according to claim 11, wherein the predetermined threshold value is configured to be a first value when the likely signal-to-noise ratio is above a first predetermined value.

13. The system according to claim 11, wherein the predetermined threshold value is configured to be a second value when the likely signal-to-noise ratio is above a second predetermined value.

14. The system according to claim 11, wherein the obtained information relates to the activity level of a person whose electrocardiogram output is being monitored.

15. The system according to claim 11, wherein the obtained information relates to a characteristic of a person whose electrocardiogram output is being monitored.

16. (canceled)

17. An electronics module comprising a controller and a memory coupled to the controller, the controller being arranged to receive signals from a sensor arrangement on a wearable article, wherein the controller is configured to obtain electrocardiogram output for a wearer of the wearable article from the sensor arrangement, the controller being further configured to:
obtain a series heartrate values for the electrocardiogram output;
determine when the rate of change of the heartrate values exceeds a predetermined threshold value; and, in response to the predetermined threshold value being exceeded,
sequentially apply one or more correcting steps to a region of the electrocardiogram output around a selected heartbeat where the rate of change of heartrate values occurred;
obtain information indicative of the likely signal-to-noise ratio of the electrocardiogram output;
configure the predetermined threshold value based on the likely signal-to-noise ratio; and
determine the rate of change of heartrate values at the selected region for each of the applied correcting steps; and
choose a correcting step corresponding to the lowest of the rate of change of heartrate values at the selected region as a permanent correction for the electrocardiogram output.

18. The electronics module according to claim 17, wherein the predetermined threshold value is configured to be a first value when the likely signal-to-noise ratio is above a first predetermined value.

19. The electronics module according to claim 17, wherein the predetermined threshold value is configured to be a second value when the likely signal-to-noise ratio is above a second predetermined value.

20. The electronics module according to claim 17, wherein the obtained information relates to the activity level of a person whose electrocardiogram output is being monitored.

21. The electronics module according to claim 17, wherein the obtained information relates to a characteristic of a person whose electrocardiogram output is being monitored.

22. The electronics module according to claim 17, wherein the controller is further configured to:
determine the rate of change of heartrate values at the selected region for each of the applied correcting steps; and
choose a correcting step corresponding to the lowest of the rate of change of heartrate values at the selected region as a permanent correction for the electrocardiogram output.

23. The electronics module according to claim 17, wherein controller is configured to derive the rate of change heart rate from the equation:
$$\vert {r}_{k}^{′} \vert = 2 \vert \frac{{t}_{k - 1} - 2 {t}_{k} + {t}_{k + 1}}{( {t}_{k - 1} - {t}_{k} ) ( {t}_{k - 1} - {t}_{k + 1} ) ( {t}_{k} - {t}_{k + 1} )} \vert$$
where rₖ′ is the rate of change of the heart rate, at the k^{th} heartbeat,
tₖ is the time of the k^{th} heartbeat, and
tₖ₋₁ and tₖ₊₁ are the times of the (k−1)^{th} and (k+1)^{th} heartbeat respectively either side of the k^{th} heartbeat.
